(19) United States
(12) Patent Application Publication
(10) Pub. No.: US 2015/0164866 A1
(43) Pub. Date: Jun. 18, 2015

(54) METHODS OF ADMINISTERING RIFAXIMIN FOR WEIGHT LOSS AND TREATMENT OF OBESITY

(71) Applicant: Salix Pharmaceuticals, Ltd, Raleigh, NC (US)

(72) Inventor: Charles W. Randall, San Antonio, TX (US)

(21) Appl. No.: 14/629,869

(22) Filed: Feb. 24, 2015

Related U.S. Application Data
(63) Continuation of application No. PCT/US2013/059589, filed on Sep. 13, 2013.
(60) Provisional application No. 61/700,866, filed on Sep. 13, 2012.

(51) Int. Cl. A61K 31/437

(52) U.S. Cl. CPC A61K 31/437

(57) ABSTRACT

Methods of reducing weight in a subject are provided, wherein the methods comprise administering a composition comprising an effective amount of rifaximin to a subject in need of treatment for weight loss. In some embodiments, the subject is considered obese (BMI>30).

### RELATED APPLICATIONS

This application is a continuation of International Application No. PCT/US2013/059589, filed Sep. 13, 2013, which claims the benefit of U.S. Provisional Application No. 61/700,866, filed on Sep. 13, 2012. The entire contents of each of the aforementioned applications are incorporated herein by reference.

### BACKGROUND

A growing body of evidence supports a relationship between obesity and changes in gut flora. The composition of the intestines is dominated by two divisions of bacteria: bacteroides and firmicutes. Obesity, dyslipidemia, hyperglycemia and other alterations in metabolism have been associated with elevated concentrations of firmicute species in both humans and animals.

Rifaximin (INN; see The Merck Index, XIII Ed., 8304) is an antibiotic belonging to the rifamycin class of antibiotics, e.g., a pyrido-imidazo rifamycin. Rifaximin exerts its broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

### SUMMARY

The instant disclosure is directed to methods of reducing weight and treating obesity in a subject in need thereof. Specifically, the methods entail administering rifaximin, or a composition comprising rifaximin, to an obese subject, or a subject in need of weight loss. Typically, the methods result in about a 2%, about 5%, or even about 10% reduction in the weight of the subject. Furthermore, subjects that can benefit by the methods described herein often have a Body Mass Index (“BMI”) of at least 25, 30, 35, or 40. The methods are useful for subjects suffering from diseases other than, or in addition to, obesity. For example, subjects may be suffering from GERD, hypertension, diabetes, or a lipid disorder.

Typically, the methods comprise administering rifaximin at a dose of about 50 mg to about 6000 mg per day. In other embodiments, rifaximin is administered at a dose of about 100 mg to about 6000 mg; from about 50 mg to about 2500 mg BID; from about 50 mg to about 2000 mg TID; 200 mg TID; 200 mg BID; or 200 mg QD. In yet other embodiments, rifaxamin is administered at a dose of about 550 mg, 600 mg or 1650 mg TID, QD or BID. Additionally, rifaxamin may be administered at a dose of about 550 mg BID.

In accordance with the methods described herein, rifaxamin may be administered throughout the duration of a subject's life. Alternatively, the rifaxamin may be administered for about 1 week to about 24 months. In some embodiments, subjects are administered rifaxamin for at least 20 days, or at least 6, 12, 24, or 36 months.

In some embodiments, the methods are directed specifically to reducing body weight comprising: identifying a subject in need of reduced body weight; administering a composition comprising rifaximin to the subject, and reducing the subject's body weight by at least 2%, 5%, or 10%. In other embodiments, the methods are directed specifically to methods of treating obesity comprising: identifying a subject in need of treatment for obesity; administering a composition comprising rifaximin to the subject; and reducing the subject's body weight by at least 2%.

In any of the foregoing embodiments, the subject can be on a sugar-restricted diet. In some embodiments, the subject's intake of free sugars is less than about 10% of the total energy intake. In some embodiments, the subject's intake of free sugars is less than about 5% of the total energy intake.

### DETAILED DESCRIPTION

Rifaximin (USAN, INN; see The Merck Index, XIII Ed., 8304, CAS No. 80621-81-4), (2S,16Z,18E,20S,21S,22R,23R,24R,25S,26S,27S,28E)-5,6,21,23,25 Pentahydroxy-27-methoxy-2,4,11,16,20,22,24,26-octamethyl-2,7-(epoxypentadeca-(1,11,13) trienimino)benzofuro (4,5-e) pyrido(1,2,-a)benzimidazole-1,15(2H)-dione,25-acetate), is a semi-synthetic antibiotic produced from rifamycin O. Rifaximin is a molecule belonging to the rifamycin class of antibiotics, e.g., a pyrido-imidazo rifamycin. Rifaximin exerts a broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency.

Rifaximin is also described in Italian Patent IT 1154655 and EP 0161534. EP patent 0161534 discloses a process for rifaximin production using rifamycin 0 as the starting material (The Merck Index, XIII Ed., 8301). U.S. Pat. No. 7,045,620 B1 discloses polymorphic forms of rifaximin, as do U.S. Ser. No. 11/658,702; U.S. Ser. No. 61/031,329; U.S. Ser. No. 12/119,622; U.S. Ser. No. 12/119,630; U.S. Ser. No. 12/119,612; U.S. Ser. No. 12/119,600; U.S. Ser. No. 11/873,841; Publication WO 2006/094662; and U.S. Ser. No. 12/393,012. The applications and patents referred to here are incorporated herein by reference in their entirety for all purposes.

Rifaximin is a compound having the structure of formula I:

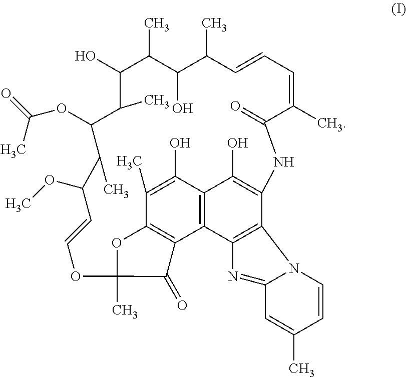 [SMILES: COC1/C=C/OC2(C)Oc3c(C)c(O)c4c(O)c(c5c(nc6cc(C)ccn65)c4c3C2=O)NC(=O)/C(C)=C\C=C\C(C)C(O)C(C)C(O)C(C)C(OC(C)=O)C1C]

Without wishing to be bound by any particular scientific theories, rifaximin acts by binding to the beta-subunit of the bacterial deoxyribonucleic acid-dependent ribonucleic acid (RNA) polymerase, resulting in inhibition of bacterial RNA synthesis. It is active against numerous gram (+) and (−) bacteria, both aerobic and anaerobic. In vitro data indicate rifaximin is active against species of Staphylococcus, Streptococcus, Enterococcus, and Enterobacteriaceae.

“Rifaximin”, as used herein, includes solvates and polymorphous forms of the molecule, including, for example, Form α, Form β, Form γ Form δ, Form ε, Form ζ, Form η, Form τ, Form kappa, Form theta, Form mu, Form omicron, Form pi, mesylate Form or amorphous Forms of rifaximin. These forms are described in more detail, for example, in EP 05 004 695.2, filed 3 Mar. 2005; U.S. Pat. No. 7,045,620; U.S. Pat. No. 7,612,199; U.S. Pat. No. 7,709,634; U.S. Pat. No. 7,915,275; U.S. Pat. No. 8,067,429; U.S. Pat. No. 8,193,196; U.S. Pat. No. 8,227,482; U.S. Pat. No. 8,383,151; U.S. Pat. No. 8,486,956; U.S. Pat. No. 8,513,275; U.S. Pat. No. 8,518,949; G. C. Viscomi, et al., CrystEngComm, 2008, 10, 1074-1081 (April 2008), and US Patent Publication 2005/0272754. Each of these references is hereby incorporated by reference in entirety.

Medicinal preparations may contain rifaximin together with standard pharmaceutical and medicinal excipients, discussed infra.

“Polymorphs” or “polymorphic forms” as used herein, refer to the occurrence of different crystalline forms of a single compound in distinct hydrate status, e.g., a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as solubility profiles, melting point temperatures, hygroscopicity, particle shape, density, flowability, compatibility and/or x-ray diffraction peaks. The solubility of each polymorph may vary, thus, identifying the existence of pharmaceutical polymorphs is essential for providing pharmaceuticals with predictable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. For a general review of polymorphs and the pharmaceutical applications of polymorphs see G. M. Wall, Pharm Manuf. 3, 33 (1986); J. K. Haleblian and W. McCrone, J Pharm. Sci., 58, 911 (1969); and J. K. Haleblian, J. Pharm. Sci., 64, 1269 (1975), all of which are incorporated herein by reference. As used herein, the term polymorph is occasionally used as a general term in reference to the forms of rifaximin and include within the context, salt, hydrate, polymorph and amorphous forms of rifaximin disclosed herein. This use depends on context and will be clear to one of skill in the art. Exemplary polymorphic forms of rifaximin useful in the methods and kits as disclosed herein are set forth in the published patent applications set forth above.

Rifaximin, or pharmaceutical and/or medicinal compositions comprising the same, can optionally be administered in combination with one or more other gastrointestinal (GI) antibiotics. A “GI specific antibiotic” (used interchangeably with “GI antibiotic”) includes an antibiotic known to have an effect on GI disease. For example, a rifamycin class antibiotic neomycin, metronidazole, teicoplanin, ciprofloxacin, doxycycline, tetracycline, augmentin, cephalexin, penicillin, ampicillin, kanamycin, rifamycin, vancomycin, and combinations thereof are useful GI specific antibiotics. In some embodiments, a GI specific antibiotic with low systemic absorption is preferred. Low systemic absorption includes, for example, less than 10% absorption, less than 5% absorption, less than 1% absorption and less than 0.5% absorption. Low systemic absorption also includes, for example, from between about 0.01-1% absorption, from between about 0.05-1% absorption, from between about 0.1-1% absorption, from between about 1-10% absorption, or from between about 5-20% absorption.

In some embodiments, rifaximin, or pharmaceutical and/or medicinal compositions comprising the same, can optionally be administered in combination with one or more other antibiotics selected from the group of: rifamycin, aminoglycoside, amphenicol, ansamycin, f3-Lactam, carbapenem, cephalosporin, cephamycin, monobactam, oxacephem, lincosamide, macrolide, tetracycline, or a 2,4-diaminopyrimidine class antibiotic.

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with rifaximin, where the untreated subjects have, or are subject to developing, the same or similar disease, condition, symptom or the like. Amelioration of a disease, condition, symptom or assay parameter may be determined subjectively or objectively, e.g., self assessment by a subject(s), by a clinician's assessment or by conducting an appropriate assay or measurement, including, e.g., weight, body-mass index (BMI), a quality of life assessment, a slowed progression of a disease(s) or condition(s), a reduced severity of a disease(s) or condition(s), or a suitable assay(s) for the level or activity(ies) of a biomolecule(s), cell(s) in a subject. Amelioration may be transient, prolonged or permanent or it may be variable at relevant times during or after rifaximin is administered to a subject or is used in an assay or other method described herein or a cited reference, e.g., within timeframes described infra, or about 1 hour after the administration or use of rifaximin to about 7 days, 2 weeks, 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with rifaximin, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively, e.g., by the subject's self assessment, by a clinician's assessment or by conducting an appropriate assay or measurement, including, e.g., weight, body-mass index (BMI), quality of life assessments or suitable assays for the level or activity of molecules within a subject. Modulation may be transient, prolonged or permanent or it may be variable at relevant times during or after rifaximin is administered to a subject or is used in an assay or other method described herein or a cited reference, e.g., within times described infra, or about 1 hour of the administration or use of rifaximin to about 2 weeks, 28 days, 3, 6, 9 months or more after a subject(s) has received rifaximin.

The term “modulate” may also refer to increases or decreases in the activity of a cell in response to exposure to rifaximin, e.g., the inhibition of proliferation and/or induction of differentiation of at least a sub-population of cells in an animal such that a desired end result is achieved, e.g., a therapeutic result of rifaximin used for treatment may increase or decrease over the course of a particular treatment.

The language “a therapeutically effective amount” or “an effective amount” of a compound refers to an amount of a compound of formula I or otherwise described herein which is effective, upon single or multiple dose administration to the subject, in producing a change in weight or BMI, such as, for example, producing a weight loss or a decrease in BMI. In some embodiments, the change in weight of the subject is at least about a 2% reduction in the weight of the subject. In some embodiments, the change in weight of the subject is at least about a 5% reduction in the weight of the subject. In some embodiments, the change in weight of the subject is at least about a 10% reduction in the weight of the subject. In some embodiments, the change in BMI of the subject is at least about a one-point drop in the subject's BMI. In some embodiments, the change in BMI of the subject is at least about a two-point drop in the subject's BMI. In some embodiments, the change in BMI of the subject is at least about a three-point drop in the subject's BMI. In some embodiments, the change in BMI of the subject is at least about a 5-, 10-, 12- or 15-point drop in the subject's BMI. In some embodiments, the change in BMI of the subject is a change to a BMI value of less than 30, 29, 28, 27, 26 or 25.

As used herein, “subject” includes organisms which are being treated with a rifamycin class antibiotic (e.g., rifaximin) for producing weight loss or a decrease in BMI, or who could otherwise benefit from the administration of a rifamycin class antibiotic (e.g., rifaximin) as described herein, such as human and non-human animals. Preferred human animals include human subjects. The term “non-human animals” includes all vertebrates, e.g., mammals, e.g., rodents, e.g., mice, and non-mammals, such as non-human primates, e.g., sheep, dog, cow, chickens, amphibians, reptiles, etc.

The term “administration” or “administering” includes routes of introducing rifaximin to a subject to perform their intended function. Examples of routes of administration that may be used include injection, oral, inhalation and rectal. The pharmaceutical preparations may be given by forms suitable for each administration route. For example, these preparations are administered in tablets or capsule form, by injection, inhalation, ointment, suppository, etc. administration by injection, infusion or inhalation; and rectal by suppositories. Oral administration is preferred. The injection can be bolus or can be continuous infusion. Depending on the route of administration, rifaximin can be coated with or disposed in a selected material to protect it from natural conditions that may detrimentally affect its ability to perform its intended function. Rifaximin can be administered alone, or in conjunction with either another agent or agents as described above or with a pharmaceutically-acceptable carrier, or both. Rifaximin can be administered prior to the administration of the other agent, simultaneously with the agent, or after the administration of the agent.

Administration “in combination with” one or more further therapeutic agents includes simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and/or the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved.

The term “obtaining” as in “obtaining rifaximin” is intended to include purchasing, synthesizing or otherwise acquiring rifaximin.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

Embodiments are directed to a method of producing weight loss in a subject, wherein the method includes administering a composition comprising an effective amount of rifaximin to the subject. In some embodiments, administration of the composition results in at least about a 2% reduction in the weight of the subject. In some embodiments, administration of the composition results in at least about a 5% reduction in the weight of the subject. In some embodiments, administration of the composition results in at least about a 10% reduction in the weight of the subject. In some embodiments, administration of the composition results in a decrease in the subject's BMI of at least about one point. In some embodiments, administration of the composition results in a decrease in the subject's BMI of at least about two points. In some embodiments, administration of the composition results in a decrease in the subject's BMI of at least about three points. In some embodiments, administration of the composition results in a decrease in the subject's BMI of at least about 5, 10, 12 or 15 points. In some embodiments, administration of the composition results in a change of the subject's BMI value to less than about 30, 29, 28, 27, 26 or 25.

In some embodiments, the subject is also suffering from a condition selected from the group of: diabetes, gastroesophageal reflux disease (GERD), hypertension, elevated cholesterol levels, a lipid disorder, a metabolic disorder, a mitochondrial disorder, an inflammatory bowel disease (IBD), travelers' diarrhea (TD), hepatic encephalopathy (HE), minimal hepatic encephalopathy, irritable bowel syndrome (IBS), diarrhea-predominant irritable bowel syndrome (d-IBS), non-constipation-predominant irritable bowel syndrome (non-c-IBS), a Clostridium difficle infection (CDI), fibromyalgia (FM), chronic fatigue syndrome (CFS), depression, attention deficit/hyperactivity disorder (ADHD), multiple sclerosis (MS), systemic lupus erythematosus (SLE), restless leg syndrome, dermal infections, small intestinal bacterial overgrowth, chronic pancreatitis, pancreatic insufficiency, diverticulitis (or diverticular disease), enteritis, colitis, skin infections, mucous membrane disorders, pouchitis, vaginal infections, anal fissures, ear infections, lung infections, periodontal conditions, rosacea, and other infections of the skin and/or other related conditions. In some embodiments, the inflammatory bowel disease is Crohn's disease or ulcerative colitis. In some embodiments, the enteritis is caused by radiation therapy or chemotherapy.

In some embodiments, a gastrointestinal (GI) cleanser is administered to a subject prior to administration of the composition.

In some embodiments, the gastrointestinal cleanser is administered between about 1 to about 90 days prior to administration of the composition. In some embodiments, the administration of the gastrointestinal cleanser is within between about 1 to about 60 days; between about 1 to about 30 days; between about 1 to about 24 days; between about 1 to about 14 days; between about 1 to about 10 days; between about 1 to about 7 days; between about 1 to about 5 days; between about 1 to about 4 days; between about 1 to about 3 days; or between about 1 to about 2 days prior to administration of the composition.

In some embodiments, the gastrointestinal cleanser comprises one or more of a PEG-based composition or a sodium phosphate-based composition. In some embodiments, the gastrointestinal cleanser comprises polyethylene glycol (PEG), sodium sulfate, sodium chloride, potassium chloride, and ascorbic acid. In some embodiments, the gastrointestinal cleanser comprises sodium phosphate monobasic, sodium phosphate dibasic, microcrystalline cellulose, colodial silicon dioxide, and magnesium stearate.

Rifaximin may be administered, for example, twice a day, three times a day, or four times or more often as necessary per day. Rifaximin may be administered in doses, for example of from about between 25 mg once daily to about 3000 mg TID. In some embodiments, the subject is administered rifaximin at a dose of about 50 mg to about 6000 mg per day. For example, rifaximin can be administered in daily doses of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, In some embodiments, rifaximin can be administered in daily doses of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, In some embodiments, rifaximin can be administered in daily doses of about 550 mg, about 600 mg, about 650 mg, about 700 mg, about 750 mg, about 800 mg, about 850 mg, about 900 mg, about 950 mg, or about 1000 mg. In some embodiments, rifaximin can be administered in daily doses of about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg, In some embodiments, rifaximin can be administered in doses of about 25 mg BID, about 30 mg BID, about 35 mg BID, about 40 mg BID, about 45 mg BID, about 50 mg BID, about 55 mg BID, about 60 mg BID, about 65 mg BID, about 70 mg BID, about 75 mg BID, about 80 mg BID, about 85 mg BID, about 90 mg BID, about 95 mg BID, or about 100 mg BID, In some embodiments, rifaximin can be administered in doses of about 125 mg BID, about 150 mg BID, about 175 mg BID, about 200 mg BID, about 225 mg BID, about 250 mg BID, about 275 mg BID, about 300 mg BID, about 325 mg BID, about 350 mg BID, about 375 mg BID, about 400 mg BID, about 425 mg BID, about 450 mg BID, about 475 mg BID, or about 500 mg BID, In some embodiments, rifaximin can be administered in doses of about 550 mg BID, about 600 mg BID, about 650 mg BID, about 700 mg BID, about 750 mg BID, about 800 mg BID, about 850 mg BID, about 900 mg BID, about 950 mg BID, or about 1000 mg BID. In some embodiments, rifaximin can be administered in doses of about 1100 mg BID, about 1200 mg BID, about 1300 mg BID, about 1400 mg BID, about 1500 mg BID, about 1600 mg BID, about 1700 mg BID, about 1800 mg BID, about 1900 mg BID, about 2000 mg BID, about 2100 mg BID, about 2200 mg BID, about 2300 mg BID, about 2400 mg BID, about 2500 mg BID, about 2600 mg BID, about 2700 mg BID, about 2800 mg BID, about 2900 mg BID or about 3000 mg BID, In some embodiments, rifaximin can be administered in doses of about 25 mg TID, about 30 mg TID, about 35 mg TID, about 40 mg TID, about 45 mg TID, about 50 mg TID, about 55 mg TID, about 60 mg TID, about 65 mg TID, about 70 mg TID, about 75 mg TID, about 80 mg TID, about 85 mg TID, about 90 mg TID, about 95 mg TID, or about 100 mg TID, In some embodiments, rifaximin can be administered in doses of about 125 mg TID, about 150 mg TID, about 175 mg TID, about 200 mg TID, about 225 mg TID, about 250 mg TID, about 275 mg TID, about 300 mg TID, about 325 mg TID, about 350 mg TID, about 375 mg TID, about 400 mg TID, about 425 mg TID, about 450 mg TID, about 475 mg TID, or about 500 mg TID, In some embodiments, rifaximin can be administered in doses of about 550 mg TID, about 600 mg TID, about 650 mg TID, about 700 mg TID, about 750 mg TID, about 800 mg TID, about 850 mg TID, about 900 mg TID, about 950 mg TID, or about 1000 mg TID. In some embodiments, rifaximin can be administered in doses of about 1100 mg TID, about 1200 mg TID, about 1300 mg TID, about 1400 mg TID, about 1500 mg TID, about 1600 mg TID, about 1700 mg TID, about 1800 mg TID, about 1900 mg TID, about 2000 mg TID, about 2100 mg TID, about 2200 mg TID, about 2300 mg TID, about 2400 mg TID, about 2500 mg TID, about 2600 mg TID, about 2700 mg TID, about 2800 mg TID, about 2900 mg TID or about 3000 mg TID, The rifaximin may be administered, for example, in tablet form, powdered form, liquid form or in capsules. In some embodiments, rifaximin can be administered in a time-released formulation.

In some embodiments, rifaximin is administered as a soluble solid dispersion. For example, rifaximin can be administered at between about 25-550 mg of soluble solid dispersion of rifaximin. Soluble solid dispersions of rifaximin are described in “FORMULATIONS OF RIFAXIMIN AND USES THEREOF,” U.S. Patent Publication No. 2012/0077835, which is incorporated herein by reference in its entirety.

In some embodiments, the rifaximin is administered to a subject from between about 1 week to about 6 weeks in duration, from between about 8 weeks to about 12 weeks in duration, or from between about 1 day to about 21 days in duration. In some embodiments, rifaximin is administered for 10 days. In some embodiments, rifaximin is administered for 20 days. The rifaximin may be administered from between about 1 day and about 1 year, or from 1 week to about 52 weeks. In some embodiments, the rifaximin is administered from between about one week and about 24 months. The rifaximin may be administered intermittently or continuously during the course of treatment. Length of treatment may vary depending on the type and length of disease and the proper length of treatment may be easily determined by one of skill in the art having the benefit of this disclosure.

For any of the embodiments, rifaximin may be administered, for example, once daily, twice daily, three times daily, or four times daily (or more often as necessary for a particular subject) to a subject. In some embodiments, the methods comprise administering the rifaximin once daily to the subject because it may, for example, minimize the side effects and increase patient compliance. In some embodiments, rifaximin is administered twice and/or three times daily.

Dosages, according to certain preferred embodiments, range from between about 50 to about 6000 mg of rifaximin administered daily. For example, a dose of 400 mg may be administered to a subject three times daily, or a dose of 550 mg may be administered to a subject twice daily. Other appropriate dosages for the methods as disclosed herein may be determined by health care professionals or by the subject. The amount of rifaximin administered daily may be increased or decreased based on the weight, age, health, sex or medical condition of the subject. One of skill in the art would be able to determine the proper dose for a subject based on this disclosure.

Embodiments of the invention also include pharmaceutical compositions comprising an effective amount of rifaximin described herein and a pharmaceutically acceptable carrier.

In some embodiments, the pharmaceutical composition comprises rifaximin or any polymorphic form thereof and a pharmaceutically acceptable carrier. That is, formulations may contain only one polymorph or may contain a mixture of more than one polymorph. Polymorph, in this context, refers to any physical form, hydrate, acid, salt or the like of rifaximin. Mixtures may be selected, for example on the basis of desired amounts of systemic adsorption, dissolution profile, desired location in the digestive tract to be treated, and the like. The pharmaceutical composition further comprises excipients, for example, one or more of a diluting agent, binding agent, lubricating agent, disintegrating agent, coloring agent, flavoring agent or sweetening agent. Compositions may be formulated for selected coated and uncoated tablets, hard and soft gelatin capsules, sugar-coated pills, lozenges, wafer sheets, pellets and powders in sealed packet. For example, compositions may be formulated for topical use, for example, ointments, pomades, creams, gels and lotions.

In some embodiments, rifaximin is administered to the subject using a pharmaceutically-acceptable formulation, e.g., a pharmaceutically-acceptable formulation that provides sustained delivery of rifaximin to a subject for at least 12 hours, 24 hours, 36 hours, 48 hours, one week, two weeks, three weeks, or four weeks after the pharmaceutically-acceptable formulation is administered to the subject.

In some embodiments, these pharmaceutical compositions are suitable for oral administration to a subject. In some embodiments, as described in detail below, the pharmaceutical compositions may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intrarectally, for example, as a pessary, cream or foam; or (4) aerosol, for example, as an aqueous aerosol, liposomal preparation or solid particles containing the compound.

The phrase “pharmaceutically acceptable” refers to rifaximin, compositions containing rifaximin, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing rifaximin include those suitable for oral, nasal, topical (including buccal and sublingual), rectal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1% to about 99% of active ingredient, preferably from about 5% to about 70%, most preferably from about 10% to about 30%.

Liquid dosage forms for oral or rectal administration of rifaximin include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to rifaximin may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions for rectal administration may be presented as a suppository, which may be prepared by mixing rifaximin with one or more suitable nonirritating excipients or carriers comprising, for example, cocoa butter, polyethylene glycol, a suppository wax or a salicylate, and which is solid at room temperature, but liquid at body temperature and, therefore, will melt in the rectum and release the active agent.

Dosage forms for the topical or transdermal administration of rifaximin can include powders, sprays, ointments, pastes, creams, lotions, gels, solutions, patches and inhalants. The rifaximin may be mixed under sterile conditions with a pharmaceutically-acceptable carrier, and with any preservatives, buffers, or propellants which may be beneficial.

The ointments, pastes, creams and gels may contain, in addition to rifaximin, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to rifaximin, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Rifaximin can be alternatively administered by aerosol. This is accomplished, for example, by preparing an aqueous aerosol, liposomal preparation or solid particles containing the compound. A non-aqueous (e.g., fluorocarbon propellant) suspension could be used. Sonic nebulizers are preferred because they minimize exposing the agent to shear, which can result in degradation of the compound.

Examples of suitable aqueous and non-aqueous carriers which may be employed in the pharmaceutical compositions can include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, to prolong the effect of a drug, it is desirable to alter the absorption of the drug. This may be accomplished by the use of a liquid suspension of crystalline, salt oramorphous material having poor water solubility. The rate of absorption of the drug may then depend on its rate of dissolution which, in turn, may depend on crystal size and crystalline form. Alternatively, delayed absorption of a drug form is accomplished by dissolving or suspending the drug in an oil vehicle.

When the rifaximin is administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, rifaximin, which may be used in a suitable hydrated form and/or pharmaceutical compositions as disclosed herein, are formulated into pharmaceutically-acceptable dosage forms by methods known to those of skill in the art.

Actual dosage levels and time course of administration of the active ingredients in the pharmaceutical compositions as disclosed herein may be varied so as to obtain an amount of the active ingredient which is effective to achieve the desired therapeutic response for a particular patient, composition, and mode of administration, without being toxic to the patient. An exemplary dose range is from 25 to 3000 mg per day.

In some embodiments, the subject is also undergoing a therapy for weight loss treatment. This can include, for example, meeting with a nutritionist, following a meal plan, restricting caloric intake, and starting or maintaining an exercise program.

In some embodiments, the subject is on a sugar-restricted diet. In some embodiments, the subject's intake of free sugars is less than about 10% of the subject's total energy intake. In some embodiments, the subject's intake of free sugars is less than about 9%, 8%, 7%, 6% or 5% of the subject's total energy take. Free sugars typically refer to all monosaccharides and disaccharides added to foods by a manufacturer, cook or consumers. Free sugars also include, for example, sugars that are naturally present in honey, syrups and fruit juices.

In some embodiments, the subject is administered a second agent in combination with rifaximin, wherein the second agent is selected from the group of: dextroamphetamine, benzphetamine, methamphetamine, phentermine hydrochloride, phendimetrazine, diethylpropion, and sibutramine. The second agent can be administered before administration of rifaximin, concurrently with administration of rifaximin, or after administration of rifaximin.

In some embodiments, rifaximin and the second agent are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart.

In some embodiments, rifaximin and the second agent are cyclically administered. Cycling therapy involves the administration of a first therapy (e.g., a first therapeutic agent) for a period of time, followed by the administration of a second therapy (e.g., a second therapeutic agent) for a period of time, optionally, followed by the administration of a third therapy (e.g., therapeutic agent) for a period of time and so forth, and repeating this sequential administration, e.g., the cycle in order to reduce the development of resistance to one of the therapies, to avoid or reduce the side effects of one of the therapies, and/or to improve the efficacy of the therapies.

In certain embodiments, the administration of the same compounds may be repeated and the administrations may be separated by at least about 1 day, 2 days, 3 days, 5 days, 10 days, 15 days, 30 days, 45 days, 3 weeks, 4 weeks, 5 weeks, 6 weeks, 12 weeks, 2 months, 75 days, 3 months, or at least 6 months. In other embodiments, the administration of the same therapy (e.g., therapeutic agent) other than rifaximin may be repeated and the administration may be separated by at least at least 1 day, 2 days, 3 days, 5 days, 10 days, 15 days, 30 days, 45 days, 2 months, 75 days, 3 months, or at least 6 months. In some embodiments, a label on a rifaximin antibiotic may instruct, for example, do not repeat more often than every 6 weeks. In some embodiments, a label on a rifaximin antibiotic may instruct, for example, do not repeat more often than every 3 weeks. In another embodiment, a label on a rifaximin antibiotic may instruct, for example, do not repeat more often than every 3-12 weeks. Included within ranges given herein for dosage or administration are any value within the range.

In some embodiments, retreatment is efficacious in combination with the methods disclosed herein. For example, methods as described herein may further comprise determining symptom relief in a subject and administering a second course of rifaximin treatment if symptoms remain unresolved.

Kits are also provided herein, for example, kits for treating weight loss or producing a decrease in BMI, with rifaximin in a subject. The kits may contain, for example, a polymorph or amorphous form of rifaximin and instructions for use. The instructions for use may contain prescribing information, dosage information, storage information, and the like.

In some embodiments, the label describes adverse events comprising one or more of infections and infestations, gastrointestinal disorders, nervous system disorders, and musculoskeletal and connective tissue disorders.

In some embodiments, the label describes a length of treatment with rifaximin, whereby a subject is selected as responding to treatment if a healthcare professional prescribes rifaximin according to the label instructions.

In some embodiments, the label describes a length of treatment with rifaximin, whereby a subject is removed from treatment if a healthcare professional prescribes rifaximin according to the label instructions.

Packaged compositions are also provided, and may comprise a therapeutically effective amount of one or more of a one or more of an amorphous form, Form α, Form β, Form γ, Form δ, Form ε, Form ζ, Form mu, Form omicron, Form kappa, Form iota, or Form η polymorph of rifaximin of rifaximin and a pharmaceutically acceptable carrier or diluent, wherein the composition is formulated for treating a subject suffering from or susceptible to a bowel disorder, and packaged with instructions to treat a subject suffering from or susceptible to a bowel disorder.

### Examples

It should be appreciated that embodiments of the invention as disclosed herein should not be construed to be limited to the example, which is now described; rather, the embodiments can be construed to include any and all applications provided herein and all equivalent variations within the skill of the ordinary artisan.

Sixty-six patients with a BMI>30 were randomized (2:1) to receive RFX 550 mg or placebo (PBO) twice daily for 20 days. Patients were followed for up to six months. The primary endpoint was weight loss at the end of the study. Laboratory assessments including a basic metabolic panel, liver enzymes, lipid profile, and HbA1c (glucose testing) were obtained pre- and post-treatment.

Baseline demographic characteristics included median age of 45 (range 18-62) years, gender (73% female) average starting weight of 238.5±77.1 lbs and BMI of 38.8±9.7. Common co-morbidities included GERD (37%), hypertension (32%), diabetes (19%), and lipid disorders (14%). During the first study visit following treatment, patients lost a mean of 1.1 and 0.7 lbs. in the RFX and PBO groups, respectively. At the end of the study, patients lost a mean of 4.5 lbs in the RFX group over a mean follow-up of 4.9 months whereas the PBO group lost 0.7 lbs over a mean followup of 4.1 months. Weight loss in the rifaximin group was statistically significant (P<0.03) when compared to baseline weight. Diabetics tended to lose more weight (−6.1 lbs), and patients with underlying lipid disorders lost the least (−0.7 lbs). Triglycerides were the only significant laboratory difference between groups (+15.9 vs. −19.0; RFX vs. PBO, p<0.04).

Obese patients (BMI>30) who received rifaximin experienced greater weight loss as compared to placebo patients in this pilot study. The most pronounced effects were noted in patients with diabetes. A larger, randomized, controlled study can be conducted to confirm this finding as well as assess the potential effects on the microbiome.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the embodiments of the invention as described herein. Such equivalents are intended to be encompassed by the following claims.

What is claimed is:

1. A method of producing weight loss in a subject, comprising administering a composition comprising rifaximin to a subject in need thereof.

2. The method of claim 1, wherein administration of the composition results in at least about a 2% reduction in the weight of the subject.

3. The method of claim 1, wherein the administration of the composition results in at least about a 5% reduction in the weight of the subject.

4. The method of claim 1, wherein the administration of the composition results in at least about a 10% reduction in the weight of the subject.

5. The method of claim 1, wherein the subject is administered rifaximin at a dose of about 50 mg to about 6000 mg per day.

6. The method of claim 1, wherein the subject is administered rifaximin at a dose of between about 100 mg and about 6000 mg; from between about 50 mg and about 2500 mg BID; from between about 50 mg and about 2000 mg TID; 200 mg TID; 200 mg BID or 200 mg QD.

7. The method of claim 1, wherein the subject is administered rifaximin at a dose of about 550 mg, 600 mg or 1650 mg TID, QD or BID.

8. The method of claim 1, wherein the subject is administered rifaximin at a dose of about 550 mg BID.

9. The method of claim 1, wherein the subject is administered the composition for between about 1 week and about 24 months.

10. The method of claim 1, wherein the subject is administered the composition for about 20 days.

11. The method of claim 1, wherein the subject has a body mass index (BMI) of greater than about 30.

12. The method of claim 1, wherein the subject is also suffering from at least one of: GERD, hypertension, diabetes, and a lipid disorder.

13. A method of reducing body weight comprising:
identifying a subject in need of reduced body weight;
administering a composition comprising rifaximin to the subject, and reducing the subject's body weight by at least 2%.

14. A method of treating obesity comprising:
identifying a subject in need of treatment for obesity;
administering a composition comprising rifaximin to the subject, and reducing the subject's body weight by at least 2%.

15. The method of claim 13, wherein the subject's body weight is reduced by at least 5% or 10%.

16. The method of claim 13, wherein the subject in need of treatment has a BMI of at least 30, 35, or 40.

17. The method of claim 13, wherein the subject is administered rifaximin at a dose of about 50 mg to about 6000 mg per day.

18. The method of claim 13, wherein the subject is administered rifaximin at a dose of about 100 mg to about 6000 mg; from about 50 mg to about 2500 mg BID; from about 50 mg to about 2000 mg TID; 200 mg TID; 200 mg BID or 200 mg QD.

19. The method of claim 13, wherein the subject is administered rifaximin at a dose of about 550 mg, 600 mg or 1650 mg TID, QD or BID.

20. The method of claim 13, wherein the subject is administered rifaximin at a dose of about 550 mg BID.

21. The method of claim 13, wherein the subject is administered the composition for between about 1 week and about 24 months.

22. The method of claim 13, wherein the subject is administered the composition for about 20 days.

23. The method of claim 1, wherein the subject is on a sugar-restricted diet.

24. The method of claim 23, wherein the subject's intake of free sugars is less than about 10% of the total energy intake.

25. The method of claim 24, wherein the subject's intake of free sugars is less than about 5% of the total energy intake.
